(19) United States
(12) Patent Application Publication
(10) Pub. No.: US 2006/0292104 A1
(43) Pub. Date: Dec. 28, 2006

(54) Transparent or translucent conditioning composition packed into transparent and/or translucent container

(76) Inventors: Susan Marie Guskey, Loveland, OH (US); Koichi Saito, Kobe (JP); Paul Eugene Rienzo, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/474,605

(22) Filed: Jun. 26, 2006

Related U.S. Application Data
(60) Provisional application No. 60/693,896, filed on Jun. 24, 2005.

(51) Int. Cl. A61K 8/81

(52) U.S. Cl. 424/70.17

(57) ABSTRACT

Disclosed are hair or skin conditioning compositions wherein the composition is a rinse-off conditioning composition having a transparent or translucent appearance and comprising from about 0.1% to about 10% of a polymer and an aqueous carrier, wherein the composition is packed in a container having a transparent and/or translucent appearance, and wherein the container has at least one depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

### CROSS REFERENCE TO RELATED APPLICATION

This application claims the benefit of U.S. Provisional Application No. 60/693,896, filed on Jun. 24, 2005.

### FIELD OF THE INVENTION

The present invention relates to hair or skin conditioning compositions wherein the composition is a rinse-off conditioning composition having a transparent or translucent appearance and comprising from about 0.1% to about 10% of a polymer and an aqueous carrier, wherein the composition is packed in a container having a transparent and/or translucent appearance, and wherein the container has at least one depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

### BACKGROUND OF THE INVENTION

A variety of conditioning compositions such as hair conditioning compositions and skin conditioning compositions have been used for a variety of substrates such as hair and skin. A common method of providing conditioning benefits is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to form a gel matrix which has a suitable rheology for conditioning compositions and which is suitable for providing a variety of conditioning benefits, especially when used for hair care products, such as slippery feel, softness and reduced tangling on wet hair and softness and moisturized feel on the dry hair.

Most of the above conditioning agents are also known to make the composition opaque. There is a need for conditioning compositions having a clear product appearance i.e., transparent or translucent product appearance while providing conditioning benefits.

Most of the above conditioning agents are also known to make the composition oily and/or greasy. Such compositions provide the consumers with long-lasting oily and/or greasy feel during or after rinsing out the composition from the hair. For consumes who desire clean product usage feel, such long-lasting oily and/or greasy feel is not desirable. Thus, there is a need for conditioning compositions which provide clean rinse feel while also providing conditioning benefits.

Furthermore, most of the above conditioning agents, especially high melting point fatty compounds, are also known to weigh down the hair when these conditioning agents are included in hair care compositions. For consumers who desire maintaining or increasing hair volume such as consumers having fine hair, weighing down the hair is not desirable. Thus, there is a need for hair conditioning compositions which do not weigh down the hair while providing conditioning benefits.

Based on the foregoing, there remains a need for conditioning compositions which provide a clear product appearance while providing conditioning benefits. There is also a need for conditioning compositions which provide clean rinse feel and/or clean usage feel while providing a clear product appearance and conditioning benefits. There is also a need for such conditioning compositions which are suitable for providing further benefits such as not weighing down the hair, while providing a clear product appearance and conditioning benefits.

None of the existing art provides all of the advantages and benefits of the present invention.

### SUMMARY OF THE INVENTION

The present invention is directed to hair or skin conditioning compositions: wherein the composition is a rinse-off conditioning composition having a transparent or translucent appearance and comprising from about 0.1% to about 10% of a polymer and an aqueous carrier;

wherein the composition is packed in a container having a transparent and/or translucent appearance; and

wherein the container has at least one depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

By the combination of transparent/translucent conditioner compositions, transparent/translucent container, and the depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof, the compositions of the present invention can efficiently communicate such benefits to consumers.

These and other features, aspects, and advantages of the present invention will become better understood from a reading of the following description, and appended claims.

### BRIEF DESCRIPTION OF THE FIGURE

While the specification concludes with claims particularly pointing out and distinctly claiming the invention, it is believed that the invention will be better understood from the following description of the accompanying figure in which:

FIG. 1 illustrates a front view of a preferred embodiment of the container.

FIG. 2 illustrates a front view of another preferred embodiment of the container.

FIG. 3 illustrates a front view of another preferred embodiment of the container.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Container

The composition of the present invention is packed into a container. The container useful herein has a transparent and/or translucent appearance. The containers useful herein include, for example, a bottle having a closure on the top of the bottle, an upside down bottle having a closure at the bottom of the bottle with or without a hanger at the top of the bottle to enable the container to be hung, a bottle with a pump dispenser, a bottle with a foaming dispenser, and a tube with a closure. When using the upside down bottle having a closure at the bottom of the bottle, it is preferred to have a function such as a valve and a restricted orifice to prevent unintended dripping of the conditioner composition. The container can have the transparent and/or translucent appearance on mainly its bottle or tube portion. The closure or pump dispense can also have transparent and/or translucent appearance, however, the closure or pump may have opaque appearance. As long as the container has a transparent and/or translucent area on its surface, the container can have opaque or non-clear area on its surface. The containers have such opaque or non-clear area at a level of preferably about 30% or less of the surface area of bottle or tube, even if the bottle and/or tube have labels and/or films on its surface.

The containers can be made of any conventional polymers, as long as the containers have a transparent and/or translucent appearance. For the transparent appearance, it is preferred to use polyethylene terephthalate. The translucent appearance can be achieved by, for example, treatments of the transparent containers, the addition of ingredients such as dyes and pearlescent agents to base polymers, the use of polypropylene and/or polyethylene which are mixed with clarifying agents, or the use of label or shrink film having a translucent appearance. The treatments include, for example, spray coating (for example, fine particles are sprayed to the surface of the container and the container is coated by the fine particles, thus, the container has a translucent appearance), sandblasting, and mold surface treatment (for example, by forming fine undulation on the surface of the mold, the container made in the mold has fine undulation on the surface, thus, the container has translucent appearance).

The container of the present invention can have a print directly on the bottle or tube, or by the use of a label or film having such prints. Such prints include, for example, brand name/logo marks, product name, product benefits, drawings, names of ingredients. The containers with or without label/film can have other features, especially aesthetic features. Such aesthetic features include, for example, textures such as orange peel-like texture, embossing, lenticular lens, scent (activated with or without scratching the label), colors such as fluorescence, metallic color (with or without 3D-like effect), holograms, frosted or matte color, gradation of color, gradation of transparency, colored transparent/translucent appearance, drawings such as flowers' and fruits' drawings which are on the back side of the container and which can be seen from the front side of the container.

Such aesthetic features can have relationships with at least one printed information such as brand names and product names for the better communication of the printed information. For example, orange peel-like texture can be used together with drawings and/or product names regarding citrus fruits. In the present invention, it is preferred that the label also has a clear appearance, i.e., transparent and/or translucent area, more preferably transparent area. Such label can be made of any conventional polymer such as polypropylene, as long as the labels have a transparent and/or translucent area. For such transparent and/or translucent label which is used by adhering to the container, it is preferred to use an adhesive agent which provides a clear appearance. Such adhesive agents can be any conventional polymer.

For consumers who prefer more elegant-looking products, containers having translucent area may be preferred than containers having only transparent area. Containers having both translucent area and transparent area may be more preferred. By having both translucent area and transparent area, the container becomes more suitable for communicating the benefits of the composition of the present invention while having a more elegant appearance. The containers having both translucent area and transparent area can be made by, for example, the use of container having both translucent area and transparent area, the use of label having both translucent area and transparent area, or the combination of the container having a translucent area has a label having a transparent area. In the combination of the container having a translucent area and the label having a transparent area, the transparent area of the label adheres on the translucent area of the container, and the container has a transparent appearance in the area which the transparent area of label adheres on. It is believed that, when the translucent area of the container is due to fine undulations on its surface, such translucent area becomes transparent by the adhesion of label having transparent area, i.e., by adhesive agents coming into the fine undulations.

Another aesthetic feature which the container/composition of the present invention could have, for example, is a color combination of the container/composition. Such color combinations include, for example: the combination of a non-colored transparent and/or translucent container and a colored transparent or translucent composition; a colored transparent and/or translucent container and a non-colored transparent or translucent composition; a colored transparent and/or translucent container and a colored transparent or translucent composition; and a non-colored transparent and/or translucent container and a non-colored transparent or translucent composition. It is believed that the use of at least one of the followings: a non-color translucent container; a colored transparent/translucent container; a colored transparent/translucent composition; and mixtures thereof, would provide long-lasting beauty appearance compared to the combination of a non-colored transparent container and a non-colored transparent/translucent composition, by hiding yellowing of compositions due to aging. Furthermore, for consumers who prefer more elegant-looking products, the combination of a container having a non-colored transparent and/or translucent appearance and a composition having a colored transparent or translucent appearance may be more preferred than the other color combinations.

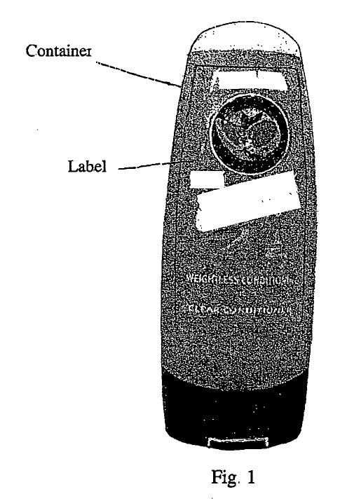

FIG. 1-3 are preferred embodiments of the containers used in the present invention.

FIG. 1 illustrates a front view of a preferred embodiment of the container which is an upside down bottle having a closure at the bottom of the bottle. The container of FIG. 1 has a non-colored translucent appearance, and has a label having a transparent area. The transparent area of the label adheres on the container, and the container has a transparent appearance in the area which the transparent area of label adheres on.

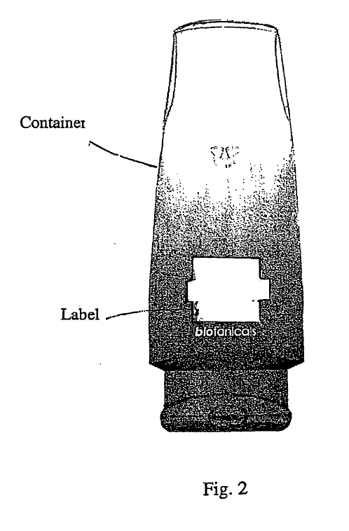

FIG. 2 illustrates a front view of another preferred embodiment of the container which is an upside down bottle having a closure at the bottom of the bottle. The container of FIG. 2 has a translucent appearance and has color gradation. The container has a label.

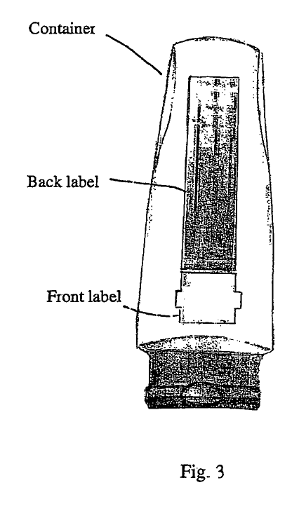

FIG. 3 illustrates a front view of another preferred embodiment of the container which is an upside down bottle having a closure at the bottom of the bottle. The container of FIG. 3 has a non-colored transparent appearance. The container of FIG. 1 has a label on both front and back sides of the container. The label on the back side has drawings such as flowers and fruits which can be seen from the front side of the container.

Depiction of Benefits

The container of the present invention has at least one depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof. Such depiction can be printed directly on the container or on a label on the container.

The depiction corresponding to “(i) Weightless hair” includes, for example, “Weightless conditioning/conditioner”, “Adds/gives volume”, “Adds/gives body”, “Adds/gives fullness”, “Does not weigh hair down”, “Airy hair”, “Uplifting/ Lifts up”, and “light weight conditioning”. This depiction corresponding to “(i) Weightless hair” is used when the composition is a hair conditioning composition, while the other depictions are used for both skin/hair conditioning compositions.

The depiction corresponding to “(ii) Light conditioning” includes, for example, “Light conditioning”, “Not heavy”, “Non-oily” “Fat-free” and “Non-creamy texture”.

The depiction corresponding to “(iii) Clean Rinse” includes, for example, “Clean rinsing feel/cleaner rinse”, “Easy/Quick to rinse”, “Rinses out quickly/easily”, “Washes away easily/quickly”, “Rinses thoroughly/completely”, and “Rinses clean”.

The depiction corresponding to “(iv) Clean Feel” includes, for example, “Without greasy residue”, “Leaves hair, skin, and/or hands feeling clean”, “No sticky feel left”, “No greasy/oily feel left”, “Does not build up”, “No coated feel”, and “Leaves hair, skin, and/or hands feeling fresh”. When the composition is a hair conditioning composition, the depiction (iv) further includes, for example, “Makes hair dry fast” and “Leaves hair feeling light/bouncy”.

The depiction corresponding to “(v) Penetrating texture” includes, for example, “Penetrates” and “Quickly absorbs”.

The depiction corresponding to “(vi) Natural” includes, for example, “Naturally conditioning”, “Pure and natural”, “Gentle to hair/skin”, “Mild to hair/skin”, “Fresh”, “Infused with vitamins, minerals and/or anti-oxidants”.

The depiction corresponding to “(vii) Refreshing and/or Energizing” includes, for example, “Refreshes”, “Purifying”, “Vitalizes/Revitalizes”, “Rejuvenates”, “Energizes”, “Renews”, “Rebirth/reborn”, “Gives hair/skin radiance”, “Restores”, and “Nourishes”.

The depiction corresponding to “(viii) Keeping style fresh” includes, for example, “Keeping style fresh all day”, and “Fresh hair style”.

Among the above depictions, the depictions (i)-(v) and (viii) are preferably used in the present invention.

Compositions

The conditioning compositions of the present invention have a clear product appearance, i.e., transparent or translucent appearance. In the present invention, the composition clear product appearance means that the composition has a transmittance of about 25% or more, preferably about 35% or more, more preferably about 40% or more, still more preferably about 50% or more, even more preferably about 60% or more. The transmittances are measured at 600 nm using UV-1601 which is a UV-visible spectrophotometer available from Shimadzu. In view of the desire for clear product appearance, it is preferred that the composition of the present invention has the above transmittance for at least one month, more preferably for at least three months, still more preferably for at least one year at 25° C., following preparation of the mixture and the composition.

In view of clear product appearance, the compositions of the present invention are preferably substantially free of substantially insoluble oily compounds. In the present invention, the compositions being “substantially free” of substantially insoluble oily compound means that the composition includes 1.0% or less, preferably 0.5% or less, more preferably 0.1% or less, still more preferably 0% of substantially insoluble oily compounds. By “substantially insoluble” oily compound, what is meant is that: the oily compound is substantially insoluble in the compositions at the level used; and the compositions has a transmittance of below about 25%, preferably below about 35%, more preferably below about 40%, still more preferably below about 50%, further more preferably below about 60% at 25° C. when containing the oily compounds at the level used. Such “substantially insoluble” oily compounds are typically those selected from hydrocarbons, fatty compounds, and mixtures thereof. Such hydrocarbons include, for example, poly α-olefin oils, paraffins, waxes, and mixtures thereof. Such fatty compounds include, for example, fatty alcohols such as cetyl alcohol and stearyl alcohol, fatty acids such as stearic acid, fatty alcohol derivatives and fatty acid derivatives such as esters and ethers thereof, and mixtures thereof.

The conditioning compositions of the present invention can be used for conditioning a variety of substrates such as hair, skin, and fabric, by applying the compositions to the substrates such as hair, skin, and fabric. The conditioning compositions of the present invention is especially suitable for hair care products such as hair conditioners, skin care products such as skin conditioners, and fabric care products such as fabric softeners.

The conditioning compositions of the present invention are especially suitable for hair conditioners for rinse-off use. Such compositions are preferably used by following steps:
(i) after shampooing hair, applying to the hair an effective amount of the conditioning compositions for conditioning the hair; and
(ii) then rinsing the hair.

The conditioning compositions of the present invention comprise a polymer and an aqueous carrier. The polymer is preferably a thickening polymer.

In a preferred embodiment (hereinafter Composition A), the conditioning composition of the present invention comprises by weight:
(a) from about 0.1% to about 10% of a thickening polymer;
(b) from about 0.1% to about 8.0% of a surfactant system selected from the group consisting of cationic surfactant, nonionic surfactant, and mixtures thereof;
(c) from about 0.1% to about 10% of a hydrophobically modified amido silicone copolyol; and
(d) an aqueous carrier;
   wherein the composition is transparent or translucent.
   Composition A preferably further contains a cationic polymer. Preferably, Composition A is substantially free of anionic compounds. Anionic compounds herein include anionic surfactants and anionic polymers. In the present invention, the compositions being “substantially free of anionic compounds” means that the compositions include 1% or less, preferably 0.5% or less, more preferably 0% of anionic compounds. Preferably, Composition A is substantially free of cationic guar polymers in view of product stability in clear product appearance. In the present invention, the composition being “substantially free of cationic guar polymers” means that the composition includes 0.1% or less, preferably 0% of cationic guar polymers.

In another preferred embodiment (hereinafter Composition B), the conditioning composition of the present invention comprises by weight:
(a) from about 0.1% to about 10% of a surfactant system comprising a cationic surfactant and a nonionic surfactant;
(b) from about 0.05% to about 10% of the polymer selected from the group consisting of an anionic polymer, an amphoteric polymer, and mixtures thereof;
(c) an aqueous carrier; and
(d) from about 0.1% to about 10% of a nonionic thickening polymer being substantially soluble in the composition;
(e) from about 0.1% to about 10% of a conditioning agent comprising a silicone compound selected from those having an average particle size of 500 nm or less in the composition, those being substantially soluble in the composition, and mixtures thereof;
   wherein the surfactant system and the polymer form a water-insoluble complex upon dilution; wherein the composition is transparent or translucent.

Composition B forms coacervates which are water-insoluble complexes. The coacervates form upon dilution of the composition, preferably, when the composition is applied to wet substrate and/or rinsed-off with water from the substrate. Preferably, coacervates form when the mass ratio of the composition to water is, preferably by about 1:50, more preferably by about 1:20, still more preferably by about 1:10. Composition B preferably further contains a co-solvent, and/or an electrolyte. Composition B may further contain a cationic polymer.

Compositions A and B are suitable for providing further benefits such as, conditioning benefits, especially softness and reduced tangling when used for hair care products such as hair conditioning products, and not weighing down the hair when used for hair care products such as hair conditioning products.

Thickening Polymer

Compositions A and B comprise a thickening polymer. The thickening polymers useful herein are those which can provide appropriate viscosity and rheology properties to the composition, so that the compositions of the present invention have: (i) a suitable viscosity of preferably from about 1,000 cps to about 150,000 cps, more preferably from about 5,000 cps to about 80,000 cps, still more preferably from about 10,000 cps to about 50,000 cps; and (ii) suitable rheology properties such that the compositions have a Shear Thinning Index (STI) of preferably about 30 or more, more preferably about 50 or more. Preferably, the composition of the present invention has the above viscosity and STI for at least one month, more preferably for at least three months, still more preferably for at least one year at 25° C., following preparation of the composition. The viscosity herein can be suitably measured by Brookfield RVT at a shear rate of 2·s⁻¹ at 26.7° C. The Shear Thinning Index (STI) is calculated according to the following equation:
Shear Thinning Index (STI)=a first viscosity/a second viscosity;
wherein the first viscosity is measured at a shear rate of 2·s⁻¹, and the second viscosity is measured at a shear rate of 950·s⁻¹, both at 26.7° C. by shear rate ramp flow measurement using AR 2000 available from TA Instruments.

The thickening agent is preferably a thickening polymer which is substantially soluble in the composition. By “substantially soluble” thickening polymer, what is meant in the present invention is that the composition has a transmittance of about 25% or more, preferably about 35% or more, more preferably about 40% or more, still more preferably about 50% or more, even more preferably about 60% or more, at 25° C. when containing the thickening polymer at he level used. The composition of the present invention preferably contain a thickening polymer at a level by weight of preferably from about 0.1% to about 10%, more preferably from about 0.1% to about 5%, still more preferably from about 0.1% to about 3%.

A variety of thickening polymers can be used in the compositions of the present invention. Thickening polymers useful herein include, for example, cellulose and its derivatives such as cellulose ethers including hydroxyethylcellulose and hydroxypropylcellulose, hydrophobically modified cellulose ethers such as cetyl hydroxyethylcellulose which is supplied, for example, by Hercules with a tradename Polysurf 67, quaternized celluloses, and hydrophobically modified cationic celluloses; guar polymers including cationic guar polymers and nonionic guar polymers such as Guar Gum 2-hydroxypropyl ether which is supplied, for example, by Rhodia with a tradename Jaguar HP-105; crosslinked polymers such as nonionic crosslinked polymers and cationic crosslinked polymers; and acrylate polymers such as sodium polyacrylate, polyethylacrylate, polyacrylamide, and hydrophobically modified crosslinked cationic acrylates. The thickening polymers useful herein may include the polymers disclosed below under the title “Cationic polymer”. Among a variety of thickening polymers, highly preferred are nonionic thickening polymers such as nonionic guar polymers, hydroxyethylcellulose, hydroxypropylcellulose, and hydrophobically modified cellulose ethers such as cetyl hydroxyethylcellulose. In Composition B, further preferred are hydroxyethylcellulose, hydroxypropylcellulose, and hydrophobically modified cellulose ethers such as cetyl hydroxyethylcellulose. In Composition B, especially preferred are hydrophobically modified cellulose ethers such as cetyl hydroxyethylcellulose.

Anionic or Amphoteric Polymer

Composition B of the present invention comprises a polymer selected from an anionic polymer, an amphoteric polymer, and mixtures thereof. The polymer is included in the compositions at a level by weight of from about 0.05% to about 10%, preferably from about 0.1% to about 5.0%, more preferably from about 0.5% to about 3.0%.

Preferably, in view of a clear product appearance, the polymer is substantially soluble in the compositions at the level used. By “substantially soluble” polymer, what is meant is that the compositions has a transmittance of about 25% or more, preferably about 35% or more, more preferably about 40% or more, still more preferably about 50% or more, even more preferably about 60% or more, at 25° C. when containing the polymer at the level used.

The polymers useful herein are those having a molecular weight of preferably 1000 AMU (Atomic Mass Unit) or more. A variety of anionic polymers and amphoteric polymers can be used in the compositions of the present invention as described below.

Anionic polymers useful herein include, for example: Polyacrylic acid; Polymethacrylic acid; Carboxyvinylpolymer; acrylate copolymers such as Acrylate/C10-30 alkyl acrylate crosspolymer, Acrylic acid/vinyl ester copolymer/Acrylates/Vinyl Isodecanoate crosspolymer, Acrylates/Palmeth-25 Acrylate copolymer, Acrylate/Steareth-20 Itaconate copolymer, and Acrylate/Celeth-20 Itaconate copolymer; sulfonate polymers such as Polysulfonic acid, Polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. Neutralizing agents may be included to neutralize the anionic polymers herein. Non-limiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof. Commercially available highly preferred anionic polymers include, for example, Carbomer supplied from Noveon under the tradename Carbopol 981 and Carbopol 980; Acrylates/C10-30 Alkyl Acrylate Crosspolymer having tradenames Pemulen TR-1, Pemulen TR-2, Carbopol 1342, Carbopol 1382, and Carbopol ETD 2020, all available from Noveon; sodium carboxymethylcellulose supplied from Hercules as CMC series; and Acrylate copolymer having a tradename Capigel supplied from Seppic. In view of clear product appearance and wet conditioning benefits, further preferred are carboxymethylcelluloses.

Amphoteric polymers useful herein include, for example, Polyquaternium-22, Polyquaternium-47, Polyquaternium-39, Octylacrylamide/Acrylates/Butylaminoethyl Methacrylate Copolymer, and Potato Starch modified. Commercially available highly preferred amphoteric polymers include, for example, Polyquaternium-39 having a tradename Merquat Plus 3330 available from Ondeo.

Surfactant System

The Compositions A and B comprise a surfactant system. The surfactant system can be included in the compositions at a level by weight of from about 0.1% to about 10%, preferably from about 0.2% to about 8%, more preferably from about 0.5% to about 5.0%.

Preferably, in view of the desire for a clear product appearance, the surfactant system is substantially soluble in the composition at the level used. By “substantially soluble” surfactant system, what is meant is that the composition has a transmittance of about 25% or more, preferably about 35% or more, more preferably about 40% or more, still more preferably about 50% or more, even more preferably about 60% or more, at 25° C. when containing the surfactant system at the level used.

Cationic Surfactant

Cationic surfactants can be included in Composition A at a level by weight of from about 0.1% to 4.0%, preferably from about 0.2% to about 3.0%, more preferably from about 0.5% to about 1.2%. Cationic surfactants can be included in Composition B at a level by weight of from about 0.1% to about 10%, preferably from about 0.25% to about 8%, more preferably from about 0.5% to about 5%.

A variety of cationic surfactants including mono- and di-alkyl chain cationic surfactants can be used in the compositions of the present invention as described below. Among them, preferred are mono-alkyl chain cationic surfactants such as mono-alkyl chain quaternary ammonium salts. The mono-alkyl chain quaternary ammonium salts useful herein are those having mono-long alkyl chain which has from 12 to 20 carbon atoms, preferably from 16 to 18 carbon atoms. Highly preferred mono-alkyl chain quaternary ammonium salts are, for example, cetyl trimethyl ammonium chloride, stearyl trimethyl ammonium chloride. Although the mono-alkyl chain cationic surfactants are preferred, other cationic surfactants such as di-alkyl chain cationic surfactants may also be used alone, or in combination with the mono-alkyl chain cationic surfactants and/or nonionic surfactants.

Cationic surfactants useful herein include, for example, those corresponding to the general formula (I): 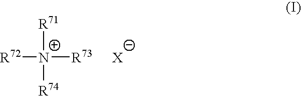
wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl.

Among the cationic surfactants of general formula (I), preferred are those containing in the molecule at least one alkyl chain having at least 16 carbons. Nonlimiting examples of such preferred cationic surfactants include: behenyl trimethyl ammonium chloride; cetyl trimethyl ammonium chloride; stearyl trimethyl ammonium chloride; olealkonium chloride; hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Also preferred are hydrophilically substituted cationic surfactants in which at least one of the substituents contain one or more aromatic, ether, ester, amido, or amino moieties present as substituents or as linkages in the radical chain, wherein at least one of the R⁷¹-R⁷⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas. Babassuamidopropalkonium Chloride available from Croda under the tradename Incroquat BA-85 is also preferably used in the composition.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethyl amine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Nonionic Surfactant

Nonionic surfactants can be included in the compositions at a level by weight of from about 0.1 to 6.0%, preferably from about 0.4% to about 5.0%, more preferably from about 1.0% to about 4.0%.

A variety of nonionic surfactants can be used in the compositions of the present invention. Among them, preferred nonionic surfactants include, for example, polyethylene glycol derivatives of glycerides, ethylene glycol ethers of fatty alcohols, and polysorbate.

Polyethylene glycol derivatives of glycerides useful herein include derivatives of mono-, di- and tri-glycerides and mixtures thereof. One class of polyethylene glycol derivatives of glycerides suitable herein is those which conform to the general formula (I):

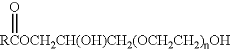

wherein n, the degree of ethoxylation, is from about 4 to about 200, preferably from about 5 to about 150, more preferably from about 20 to about 120, and wherein R comprises an aliphatic radical having from about 5 to about 25 carbon atoms, preferably from about 7 to about 20 carbon atoms. Suitable polyethylene glycol derivatives of glycerides can be polyethylene glycol derivatives of hydrogenated castor oil. Such polyethylene glycol derivatives of hydrogenated castor oil include, for example, PEG-20 hydrogenated castor oil, PEG-30 hydrogenated castor oil, PEG-40 hydrogenated castor oil, PEG-45 hydrogenated castor oil, PEG-50 hydrogenated castor oil, PEG-54 hydrogenated castor oil, PEG-55 hydrogenated castor oil, PEG-60 hydrogenated castor oil, PEG-80 hydrogenated castor oil, and PEG-100 hydrogenated castor oil. Other suitable polyethylene glycol derivatives of glycerides can be polyethylene glycol derivatives of stearic acid. Such polyethylene glycol derivatives of stearic acid include, for example, PEG-30 stearate, PEG-40 stearate, PEG-50 stearate, PEG-75 stearate, PEG-90 stearate, PEG-100 stearate, PEG-120 stearate, and PEG-150 stearate.

Ethylene glycol ethers of fatty alcohols useful herein include any ethylene glycol ethers of fatty alcohols which are suitable for use in a hair conditioning composition. No limiting examples of the ethylene glycol ethers of fatty alcohols include; the ceteth series of compounds such as ceteth-1 through ceteth-45, preferably ceteth-7 through ceteth-20; the isoceteth series of compounds such as isoceteth-20; the steareth series of compounds such as steareth-1 through 100; ceteareth 1 through ceteareth-50; the laureth series of compounds, preferably laureth-7 through Laureth-12; the pareth series of compounds, preferably pareth-9 through pareth-15; propylene glycol ethers of the above ceteth, steareth, ceteareth, and laureth series of compounds, such propylene glycol ethers of ceteth series of compounds including, for example, PPG-5-Ceteth-20; polyoxyethylene ethers or polyoxyethylene-polyoxypropylene ethers of branched alcohols, such branched alcohols including, for example, octyldodecyl alcohol, decyltetradecyl alcohol, dodecylpentadecyl alcohol, hexyldecyl alcohol, and isostearyl alcohol, and such polyoxyethylene-polyoxypropylene ethers of branched alcohols including, for example, POE(20)POP(6) decyltetradecyl ether; and mixtures thereof.

Polysorbates useful herein include, for example, polysorbate-20 (POE(20) sorbitan monolaurate) having HLB value of 16.7, polysorbate-21 (POE(4) sorbitan monolaurate) having HLB value of 13.3, polysorbate-40 (POE(20) sorbitan monopalmitate) having HLB value of 15.6, polysorbate-60 (POE(20) sorbitan monostearate) having HLB value of 14.9, polysorbate-61 (POE(4) sorbitan monostearate) having HLB value of 9.6, polysorbate-80 (POE(20)sorbitan monooleate) having HLB value of 15.0, and polysorbate-81 (POE(4) sorbitan monooleate) having HLB value of 10.0.

Preferably, the nonionic surfactants useful herein have an HLB value of from about 8 to about 28, more preferably from about 11 to about 20, still preferably from about 13 to about 18.

Among a variety of nonionic surfactants described above, highly preferred are those selected from the group consisting of isoceteth-20, PPG-5-Ceteth-20, PEG-40 hydrogenated castor oil, polysorbate-20, laureth-20, ceteth-10, ceteth-20, pareth-9, and mixtures thereof.

Conditioning Agent/Hydrophibically Modified Amido Silicone Copolyols

Composition B of the present invention contains a conditioning agent such as silicone compounds including hydrophobically modified amido silicone copolyols. Composition A of the present invention contains a hydrophobically modified amido silicone copolyols. The conditioning agents/hydrophobically modified amido silicone copolyols can be included in the compositions at a level by weight of from about 0.1% to about 10%, preferably from about 0.5% to about 8%, more preferably from about 1% to about 5%.

In view of a clear product appearance, preferred conditioning agents for Composition B are those selected from the group consisting of: (i) conditioning agent emulsions having an average particle size of 500 nm or less, preferably 300 nm or less, more preferably 100 nm or less when contained in the composition; (ii) conditioning agents being substantially soluble in the composition; and (iii) mixtures thereof. By “substantially soluble” conditioning agents, what is meant is that the compositions has a transmittance of about 25% or more, preferably about 35% or more, more preferably about 40% or more, still more preferably about 50% or more, even more preferably about 60% or more, at 25° C. when containing the conditioning agents at the level used.

In view of a clear product appearance, preferred conditioning agents for Composition B are those selected from the group consisting of (i) silicone emulsions having an average particle size of 500 nm or less, preferably 300 nm or less, more preferably 100 nm or less when contained in the composition, (ii) silicone compounds being substantially soluble in the composition, and (iii) mixtures thereof. More preferred are silicone compounds being substantially soluble in the composition. By “substantially soluble” silicone compound, what is meant is that the compositions having a transmittance of about 25% or more, preferably about 35% or more, more preferably about 40% or more, still more preferably about 50% or more, even more preferably about 60% or more, at 25° C. when containing the silicone compound at the level used.

Commercially available silicone emulsions useful herein include, for example, that with a tradename Silicone DC-8177, DC-1870, DC8168, DC8194 and DC7113 available from Dow Corning; quaternized silicone emulsion with a tradename DC5-7133 available from Dow Corning; and amodimethicone emulsion with a tradename XS65-B6413 and SME253 available from General Electric and ADM8020 available from Wacker. Such silicone emulsions useful for Composition B may contain a certain level of anionic surfactants. When such silicone emulsion containing anionic surfactants is used in Composition B, it is preferred that Composition B include 2% or less, more preferably 1% or less of anionic surfactants.

With respect to substantially soluble silicone compounds, for example, following materials can be substantially soluble depending on the level of hydrophilic groups in their structure: silicone copolyols such as dimethicone copolyols; amino silicones such as those having a amine content which is high enough to make the amino silicones substantially soluble; amino silicone copolyols such as those having an INCI name Bis (C13-15 Alkoxy) PG Amodimethicone available with a tradename DC2-8500 from Dow Corning; hydrophobically modified amino silicone copolyols; hydrophobically modified amido silicone copolyols such as those having an INCI name PEG-12 Methyl Ether/Lauroxy PEG-5 Amidopropyl Dimethicone available from Dow Corning; and quaternized silicones.

Among the substantially soluble silicone compounds, hydrophobically modified amido silicone copolyols are preferred in view of providing conditioning benefits while providing a clear product appearance. Among the hydrophobically modified amido silicone copolyols, highly preferred are those having from about 20% to about 60%, more preferably from about 25% to about 50%, still more preferably from about 30% to about 40% of ethoxylations, in view of the stability of transmittance and the stability of viscosity/STI. The percentage of the ethoxylation is calculated according to the following equation: 100×(molecular weight of ethoxyl groups)/(Molecular weight of the silicone compound).

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 40% to about 98%, and more preferably from about 50% to about 98% water.

The pH of the present compositions are preferably from about 2 to about 8, more preferably from about 3 to about 7, still more preferably from 4 to 6. Buffers and other pH adjusting agents can be included to achieve the desirable pH.

Cationic Polymer

Composition A preferably includes a cationic polymer. Composition B may also include a cationic polymer. The cationic polymers hereof will generally have a weight average molecular weight which is at least about 5,000 AMU, typically at least about 10,000 AMU, and is less than about 50 millionAMU, typically less than about 10 millionAMU, preferably, the molecular weight is from about 100,000 AMU to about 5 millionAMU, more preferably from about 500,000 AMU to about 3 millionAMU. The cationic polymers useful herein have a cationic charge density of preferably from about 0.05 meq/g to about 4.5 meq/g, more preferably about 0.1 meq/g to about 4.5 meq/g, still more preferably about 0.5 meq/g to about 4.5 meq/g.

The cationic polymer can be included in the compositions at a level by weight of preferably from about 0.05% to about 5%, more preferably from about 0.1% to about 3%, still more preferably from about 0.5% to about 2%. Preferably, in view of the desire for a clear product appearance, the cationic polymer is substantially soluble in the compositions at the level used. By “substantially soluble” cationic polymer, what is meant is that the compositions has a transmittance of about 25% or more, preferably about 35% or more, more preferably about 40% or more, still more preferably about 50% or more, even more preferably about 60% or more, at 25° C. when containing the cationic polymer at the level used.

Suitable cationic conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquatemium 6 and Polyquatemium 7, Polyquaternium-7 including that commercially available with tradenames Merquat 550 and Merquat S from Ondeo Nalco; polymethacrylamidopropyl trimonium chloride such as that commercially available with a tradename Polycare 133 from Rhone-Poulenc; and Polyquatemium-37 available from 3V Sigma with tradenames Synthalen CR, Synthalen CU, and Synthalen CN.

Also suitable cationic conditioning polymers herein include cationic cellulose derivatives. Cationic cellulose derivative useful herein include, for example, salts of hydroxyethyl cellulose reacted with trimethyl ammonium substituted epoxide, referred to in the industry (CTFA) as Polyquatemium 10, available from Amerchol Corp. (Edison, N.J., USA) in their Polymer JR® and KG® series, and also available from National Starch & Chemical with a tradename Celquat SC-230M; and Polyquatemium-4 with tradename Celquat H-100 available from National Starch & Chemical.

Cationic guar polymers, such as guar hydroxypropyltrimonium chloride commercially available from Rhodia in their Jaguar series, can also be used in the present composition. However, in Composition A, it is preferred that the compositions are substantially free of cationic guar polymers in view of product stability in clear product appearance. In the present invention, the composition being “substantially free of cationic guar polymers” means that the composition includes 0.1% or less, preferably 0% of cationic guar polymers.

Co-Solvent

Composition B preferably contains a co-solvent to help the components such as coacervates, surfactants, and silicone compounds if included, to be substantially soluble in the composition. The co-solvents useful herein are selected from the group consisting of polyhydric alcohols, water soluble alkoxylated nonionic polymers, water-soluble alkyl alcohols and ethers and mixtures thereof. The co-solvents herein can be used at levels by weight of the compositions of preferably from about 0.1% to about 20%, more preferably from about 0.5% to about 20%, still more preferably from about 1% to about 10%.

Polyhydric alcohols useful herein include, for example, glycerin, sorbitol, propylene glycol, butylene glycol, hexylene glycol, ethoxylated glucose, 1,2-hexane diol, hexanetriol, 1,5-pentane diol, dipropylene glycol, erythritol, trehalose, diglycerin, xylitol, maltitol, maltose, glucose, fructose, sodium chondroitin sultate, sodium hyaluronate, sodium adenosin phosphate, sodium lactate, pyrrolidone carbonate, glucosamine, cyclodextrin, octyne diol, diethylene glycol, and mixtures thereof.

Water soluble alkoxylated nonionic polymers useful herein include, for example, polyethylene glycols and polypropylene glycols having a molecular weight of up to about 10,000 AMU such as those with CTFA names PEG-4, PEG-8, PEG-12, PEG-20, PEG-150 and mixtures thereof.

Water soluble alkyl alcohols useful herein include, for example, monohydric C1-C6 alkyl alcohols such as ethanol, isopropyl alcohol, propanol and benzyl alcohol. Water soluble ethers useful herein include, for example, 2-butoxy ethanol, monomethyl ether of diethylene glycol, monoethyl ether of diethylene glycol, monobutyl ether of diethylene glycol.

Among a variety of co-solvents, preferred are 1,2-hexane diol, hexylene glycol, butylene glycol, glycerine, isopropyl alcohol, ethanol, propylene glycol, 1,5-pentane diol, and mixtures thereof.

Electrolyte

Composition B preferably contains an electrolyte to prevent coacervate formation in the composition prior to dilution. The electrolyte can be used at levels by weight of the compositions of, preferably from about 0.05% or more, more preferably from about 0.1% or more, still more preferably 0.5% or more in view of clear product appearance, and preferably about 10% or less, more preferably about 5.0% or less, still more preferably 3.0% or less in view of coacervate formation upon dilution.

The electrolytes useful herein are salts, and such salts useful herein include, for example, chlorides, bromides and nitrates of alkali metals, alkaline earth metals and ammonium. Preferred salts are selected from the group consisting of sodium chloride, sodium bromide, sodium nitrate, potassium chloride, potassium bromide, calcium chloride, magnesium chloride, and ammonium chloride, and mixtures thereof.

Other Additional Components

The compositions of the present invention may include additional components, which may be selected by the artisan according to the desired characteristics of the final product and which are suitable for rendering the compositions more cosmetically or aesthetically acceptable or to provide them with additional usage benefits.

The compositions of the present invention may further include other additional components. Other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, nonionic surfactants such as glyceryl stearate available from Stepan Chemicals, hydrolysed keratin, proteins, plant extracts, fruit extract, and nutrients; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate; antidandruff agents such as zinc pyrrithione, climbazol and salicylic acid; visible particles with tradenames Unisphere and Unicerin available from Induchem AG (Switzerland); and anti-foaming agent such as that with a tradename XS63-B8929 available from GE-Toshiba Silicone.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Composition A (wt %)

| | Ex. 1 | Ex. 2 | Ex. 3 | Ex. 4 | Ex. 5 | Ex. 6 | Ex. 7 |
|---|---|---|---|---|---|---|---|
| Nonionic thickening polymer-1 *1 | 1.2 | 1.4 | — | — | — | 0.5 | 1.2 |
| Nonionic thickening polymer-2 *2 | — | — | 0.5 | 2.0 | 1.2 | — | — |
| Cationic conditioning polymer-1 *3 | 0.7 | — | 1.5 | — | 0.7 | 1.5 | — |
| Cationic conditioning polymer-2 *4 | — | 0.7 | — | 0.5 | — | — | 0.5 |
| Cationic conditioning polymer-3 *5 | — | — | — | — | — | — | 0.5 |
| Cetyltrimethylammonium chloride | 1.0 | 1.0 | 1.0 | 1.0 | 1.0 | 1.0 | 1.0 |
| Stearyltrimethylamonium Chloride | — | — | — | — | — | 0.25 | — |
| C12-14 Pareth-9 *6 | — | — | 3.0 | — | — | 3.0 | — |
| Hydrophobically modified amido | 2.0 | 2.0 | — | — | — | 1.0 | — |
| silicone copolyol-1 *7 | | | | | | | |
| Hydrophobically modified amido | — | — | — | 0.5 | 2.0 | — | 2.0 |
| silicone copolyol-2 *8 | | | | | | | |
| Methylchloroisothiazolinone/ | 0.025 | 0.025 | 0.025 | 0.025 | 0.025 | 0.025 | 0.025 |
| Methylisothiazolinone *9 | | | | | | | |
| Methyl Paraben | 0.2 | 0.2 | 0.2 | 0.2 | 0.2 | 0.2 | 0.2 |
| Disodium EDTA | 0.1 | 0.1 | 0.1 | 0.1 | 0.1 | 0.1 | 0.1 |
| Perfume | 0.6 | 0.7 | — | 0.3 | 0.7 | 0.1 | 0.5 |
| Dye | 0.0003 | 0.0003 | — | — | 0.0003 | — | — |

| | |
|---|---|
| Deionized Water | q.s. to 100% |

Definitions of Components for Composition A
*1 Nonionic thickening polymer-1: Jaguar HP-105 having a molecular weight of about 2,000,000 AMU available from Rhodia
*2 Nonionic thickening polymer-2: Hydroxyethyl Ethylcellulose having a tradename Elfacos CD 481 available from AKZONOBEL
*3 Cationic conditioning polymer-1: Polyquaternium-10 having a tradename Polymer JR30M available from Amerchol
*4 Cationic conditioning polymer-2: Polyquaternium-4 having a tradename Celquat H100 available from National Starch
*5 Cationic conditioning polymer-3: Jaguar Excel available from Rhodia
*6 C12-14 Pareth-9: BT-9 available from Nikkol
*7 Hydrophobically modified amido silicone copolyol-1: PEG-12 Methyl Ether/Lauroxy PEG-5 Amidopropyl Dimethicone having 22% of ethoxylation
*8 Hydrophobically modified amido silicone copolyol-2: PEG-12 Methyl Ether/Lauroxy PEG-5 Amidopropyl Dimethicone having 24% of ethoxylation
*9 Methylchloroisothiazolinone/Methylisothiazolinone: Kathon CG available from Rohm&Haas

Method of Preparation for Composition A

The conditioning compositions of “Ex.1” to “Ex.7” as shown above can be prepared by any conventional method well known in the art. They are suitably made as follows:

The polymeric materials are dispersed in water at room temperature, mixed with vigorous agitation, and heated to 50-70° C. Cationic surfactants, and if included, nonionic surfactants, humectants, and other temperature insensitive components are added to the mixture with agitation. Then the mixture is cooled down to below 40° C., and then the remaining components such as silicones, perfumes, preservatives, and anti-foaming agents, if included, are added to the mixture with agitation.

Compositions B (wt %)

| | Ex. 8 | Ex. 9 | Ex. 10 | Ex. 11 | Ex. 12 | Ex. 13 |
|---|---|---|---|---|---|---|
| Anionic polymer-1 *1 | — | — | — | 0.25 | — | — |
| Anionic polymer-2 *2 | | | — | — | — | 2.0 |
| Anionic polymer-3 *3 | 0.25 | 1.0 | 1.0 | — | 2.0 | — |
| Cationic surfactant-1 *4 | 0.5 | | — | 1.0 | — | — |
| Cationic surfactant-2 *5 | | 1.0 | 1.0 | — | 1.0 | 1.0 |
| Nonionic surfactant-1 *6 | — | 2.5 | 2.5 | — | 2.5 | 2.5 |
| Nonionic surfactant-2 *7 | 2.5 | — | — | 1.5 | — | — |
| Thickening polymer-1 *8 | 1.8 | — | — | 1.2 | — | — |
| Thickening polymer-2 *9 | — | 1.5 | 1.4 | — | 0.8 | — |
| Thickening polymer-3 *10 | — | — | — | 1.8 | — | — |
| Thickening polymer-4 *11 | — | — | — | — | — | 0.5 |
| Cationic polymer*12 | 0.5 | — | — | — | — | — |
| Dimethicone copolyol*13 | 0.5 | — | — | 0.5 | — | — |
| Hydrophobically modified amidomethicone | — | 2.0 | 2.0 | — | 1.0 | 1.0 |
| copolyo l*14 | | | | | | |
| Ethanol | — | 3.0 | 3.0 | — | 3.0 | 3.0 |
| Isopropyl alcohol | 3.0 | | — | 3.0 | — | — |
| Methylchloroisothiazolinone/ | 0.001 | 0.001 | 0.001 | 0.001 | 0.001 | 0.001 |
| Methylisothiazolinone*15 | | | | | | |
| NaOH | — | — | — | 0.17 | — | 1.36 |
| Sodium Chloride | 1.0 | 0.6 | 0.8 | 1.0 | 0.5 | 1.5 |
| Disodium EDTA | 0.13 | 0.13 | 0.13 | 0.13 | 0.13 | 0.13 |
| m-Paraben | 0.2 | 0.2 | 0.2 | 0.2 | 0.2 | 0.2 |
| Perfume | 0.7 | 0.7 | 0.7 | 0.1 | 0.7 | 0.7 |

| | |
|---|---|
| Deionized water | q.s. to 100% |

Definitions of Components for Composition B
*1 Anionic polymer-1: Carbomer having a tradename Carbopol 980 available from Noveon
*2 Anionic polymer-2: Carbomer having a tradename Carbopol 981 available from Noveon
*3 Anionic polymer-3: Sodium Carboxymethylcellulose having a tradename CMC7M31F available from Hercules
*4 Cationic surfactant-1: Cetrimonium Chloride
*5 Cationic surfactant-2: Stearyltrimonium Chloride
*6 Nonionic surfactant-1: Ceteth-20 having a tradename Nikkol BC-20TX available from Nikko Chemicals
*7 Nonionic surfactant-2: Laureth-9
*8 Thickening polymer-1: Guar Gum 2-Hydroxypropyl Ether having a tradename Jaguar HP-105 available from Rhodia
*9 Thickening polymer-2: Cetyl hydroxyethyl cellulose having a tradename Polysurf 67 available form Hercules.
*10 Thickening polymer-3: Hydroxyethyl cellulose having a tradename Natrosol from Hercules
*11 Thickening polymer-4: Hydroxypropyl cellulose having a tradename Klucel from CP Kelco
*12 Cationic polymer: Polyquaternium-7 available from Ondeo Nalco with a tradename Merquat S
*13 Dimethicone copolyol: Silsoft 810 available from GE Silicone
*14 Hydrophobically modified amidomethicone copolyol: PEG-12 Methyl Ether/Lauroxy PEG-5 Amidopropyl Dimethicone having 20-60% of ethoxylation available from Dow Corning
*15 Methylchloroisothiazolinone/Methylisothiazolinone: Kathon CG available from Rohm&Haas

Method of Preparation for Composition B

The conditioning compositions of “Ex.8” to “Ex.13” as shown above can be prepared by any conventional method well known in the art. They are suitably made as follows:

Nonionic surfactants are added in water at room temperature, and polymeric materials are added and mixed with vigorous agitation. Then, cationic surfactants, co-solvents, electrolytes, silicone compounds, and other remaining components such as preservatives and perfumes are added, with or without pre-mixing, into the mixture of the nonionic surfactants and polymeric materials with agitation. Alternatively, refractory materials such as m-paraben can be added into the mixture of the nonionic surfactants and polymeric materials at about 60° C. to dissolve such refractory materials. In such case, the mixture is cooled to about 25° C., then cationic surfactants, co-solvents, electrolytes, silicone compounds, and other remaining components such as perfumes are added, with or without pre-mixing, into the mixture with agitation.

Examples 1 through 13 are conditioning compositions of the present invention which are particularly useful for hair conditioners for rinse-off use. The compositions of Examples 1 through 13 have a transparent or translucent appearance. The compositions of “Ex.1” through “Ex.13” have many advantages. For example, the compositions of “Ex.1” through “Ex.13” can provide conditioning benefits, especially softness and reduced tangling. The compositions of “Ex.1” through “Ex.13” can provide the above benefits while not weighing down the hair. The compositions of “Ex.1” through “Ex.13” can the above benefits while providing clean rinse feel and/or clean usage feel.

The compositions of Examples 1, 3, 4, 8, and 9 are packed into a container of FIG. 1. On the label of the container, there are depictions corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

The compositions of Examples 2, 6, 10 and 11 are packed into a container of FIG. 2. On the label of the container, there are depictions corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

The compositions of Examples 5, 7, 12 and 13 are packed into a container of FIG. 3. On the label of the container, there are depictions corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

By the combination of transparent/translucent conditioner compositions, transparent/translucent container, and the depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof, the compositions of the present invention can efficiently communicate such benefits to consumers.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair or skin conditioning composition:
wherein the composition is a rinse-off conditioning composition having a transparent or translucent appearance and comprising from about 0.1% to about 10% of a polymer and an aqueous carrier;
wherein the composition is packed in a container having a transparent and/or translucent appearance; and
wherein the container has at least one depiction corresponding to at least one of the following benefits: (i) Weightless hair, (ii) Light conditioning, (iii) Clean rinse, (iv) Clean feel, (v) Penetrating texture, (vi) Natural, (vii) Refreshing and/or Energizing, (viii) Keeping style fresh and (ix) mixtures thereof.

2. The conditioning composition of claim 1 wherein the depiction corresponds to at least one of the following benefits: (i) Weightless hair, (ii) Light conditioning, (iii) Clean rinse, (iv) Clean feel, (v) Penetrating texture, and mixtures thereof.

3. The conditioning composition of claim 1 wherein the container has a translucent area.

4. The conditioning composition of claim 3 wherein the container also has a transparent area.

5. The conditioning composition of claim 4 wherein the container having a translucent area has a label having a transparent area, wherein the transparent area of the label adheres on the translucent area of the container, wherein, in the area which the transparent area of label adhere on, the container has a transparent appearance.

6. The conditioning composition of claim 1 wherein the container has a non-colored transparent and/or translucent appearance, and wherein the composition has a colored transparent or translucent appearance.

7. The conditioning composition of claim 1 comprising by weight:
(a) from about 0.1% to about 10% of a thickening polymer system;
(b) from about 0.1% to about 10% of a surfactant system selected from the group consisting of cationic surfactant, nonionic surfactant, and mixtures thereof;
(c) from about 0.1% to about 10% of a hydrophobically modified amido silicone copolyol; and
(d) an aqueous carrier;
wherein the composition is transparent or translucent.

8. The conditioning composition of claim 7 further comprising from about 0.05% to about 5.0% of a cationic polymer.

9. The conditioning composition of claim 7 wherein the composition is substantially free of cationic guar polymers.

10. The conditioning composition of claim 1 comprising by weight:
(a) from about 0.1% to about 10% of a surfactant system comprising a cationic surfactant and a nonionic surfactant;
(b) from about 0.05% to about 10% of the polymer selected from the group consisting of an anionic polymer, an amphoteric polymer, and mixtures thereof;
(c) an aqueous carrier; and
(d) from about 0.1% to about 10% of a nonionic thickening polymer being substantially soluble in the composition;
(e) from about 0.1% to about 10% of a conditioning agent comprising a silicone compound selected from those having an average particle size of 500 nm or less in the composition, those being substantially soluble in the composition, and mixtures thereof;
wherein the surfactant system and the polymer form a water-insoluble complex upon dilution; wherein the composition is transparent or translucent.

11. The conditioning composition of claim 10 further comprising from about 0.1% to about 20% of a co-solvent.

12. The conditioning composition of claim 10 further comprising from about 0.05% to about 10% of an electrolyte.

13. The conditioning composition of claim 10 wherein the conditioning agent is the substantially soluble silicone compound being a hydrophobically modified amido silicone copolyol.

14. The conditioning composition of claim 7 wherein the composition is substantially free of substantially insoluble oily compounds.

15. The conditioning composition of claim 10 wherein the composition is substantially free of substantially insoluble oily compounds.

16. The conditioning composition of claim 7 wherein the hydrophobically modified amido silicone copolyols are those having from about 20% to about 60% of ethoxylation.

17. The conditioning composition of claim 13 wherein the hydrophobically modified amido silicone copolyols are those having from about 20% to about 60% of ethoxylation.

18. The conditioning composition of claim 1 which has a transmittance of about 25% or more.

19. The conditioning composition of claim 1 which is a hair conditioning composition.

20. A method of conditioning hair, the method comprising following steps:
(i) after shampooing hair, applying to the hair an effective amount of the conditioning composition of claim 1 for conditioning the hair; and
(ii) then rinsing the hair.
